(19) United States
(12) Patent Application Publication
(10) Pub. No.: US 2025/0090375 A1
(43) Pub. Date: Mar. 20, 2025

(54) EYE DROP DISPENSING DEVICE

(71) Applicant: AKRIVISION TECHNOLOGIES, TRUCHTERSHEIM (FR)

(72) Inventors: Philippe DAULL, Soisy Sur Seine (FR); Pierre ROY, Paris (FR)

(21) Appl. No.: 18/728,285

(22) PCT Filed: Jan. 12, 2023
(86) PCT No.: PCT/FR2023/000004

§ 371 Date: Jul. 11, 2024

(30) Foreign Application Priority Data
Jan. 14, 2022 (FR) 2200304

(51) Int. Cl. A61F 9/00

(52) U.S. Cl. CPC A61F 9/0026

(57) ABSTRACT

The application relates to an eye drop dispensing device optionally including one or more active principles, the device including a dispensing head including an eye drop supply duct, the supply duct having an open dispensing end, and a dispensing surface in the vicinity of the dispensing end, the dispensing surface extending over at least one side of and below the dispensing end, the dispensing end being arranged so as to supply the eye drops to the dispensing surface.

### TECHNICAL FIELD OF THE INVENTION

The invention relates to the field of devices for dispensing an eyedrop, optionally having one or more active principles.

### PRIOR ART

At present, the vast majority of devices for dispensing an eyedrop are designed such that the patient user has to tilt their head back and hold the eyedrop dispenser so that the delivery end of the dispenser is substantially above the target eye of the patient. Once the dispenser is in this position, the patient generally squeezes the body of the vial associated with the dispensing device in order to cause an eyedrop to fall into the target eye. This usually leads to poor dispensing of the eyedrop solution in terms of the prescribed dosage: too much eyedrop solution applied to the target eye, or the eyedrop missing the target eye, not to mention the inconvenience for the patient performing the procedure, even if the latter calls on a third person to carry it out. To date, there is no eyedrop dispenser on the market that allows the patient to put an eyedrop into the eye without tilting their head back. Dispensing aids do exist, but their designs also require the patient to tilt the head back in order to put an eyedrop into the eye when using these in combination with the existing eyedrop dispensers.

In order to overcome some of these drawbacks, the document U.S. Pat. No. 6,869,421 describes an eyedrop dispenser for non-gravity presentation of the eyedrop. To do this, the dispensing device of this document has a presentation surface at the bottom of which there open out a series of capillary tubes for supplying the eyedrop solution from the vial and for forming the droplet at the presentation surface and for maintaining the droplet in place until a predetermined dispensing inclination is reached, starting from which the droplet is deposited or falls onto the surface of the patient's target eye. The dispensing is effected by tilting the patient's head. However, the maneuver remains awkward for the patient.

### DESCRIPTION OF THE INVENTION

An object of the invention is to make available an eyedrop dispenser which does not have the aforementioned drawbacks, in particular which allows an eyedrop to be dispensed according to the prescribed dosage, without the patient having to tilt their head back and raise their arm above the head.

To this end, the invention provides a device for dispensing an eyedrop, optionally having one or more active principles, comprising a dispensing head having a supply conduit for eyedrop solution, the supply conduit having an open delivery end, and a dispensing surface in proximity to the delivery end, the dispensing surface extending on at least one side of and below the delivery end, the device being in a vertical position, the dispensing head at the top, the delivery end being arranged to generate the eyedrop toward the dispensing surface, thereby allowing the patient to place the eyedrop in an eye without having to tilt the head back.

Advantageously, but optionally, the device according to the invention for dispensing an eyedrop has at least one of the following technical features:
the delivery end is asymmetrical with respect to a longitudinal axis of the device;
the delivery end has an opening inclined toward the dispensing surface;
the inclined opening has an inclined surface;
the supply conduit has a distal portion inclined with respect to a longitudinal axis of the device, the delivery end being positioned on the inclined distal portion;
the supply conduit comprises an attachment buffer surface situated at the proximal end and arranged to permit retention of a drop and to calibrate the drop;
the supply conduit has means forming a delivery pump;
the means forming a delivery pump comprise a peristaltic pump;
the supply conduit comprises an antibacterial filter;
the supply conduit comprises a non-return valve;
the dispensing surface comprises a dispensing rim opposite the delivery end;
the dispensing surface has a flat bottom, in the form of an ovoid portion or a portion of a sphere; and
the dispensing surface is hydrophobic.

### BRIEF DESCRIPTION OF THE FIGURES

Other features and advantages of the invention will emerge from reading the following description of an embodiment of the invention. In the attached drawings:

FIG. 1 is a schematic sectional view of a first embodiment of a device according to the invention for dispensing an eyedrop;

FIG. 2 is a partial three-dimensional view of the dispensing device of FIG. 1;

FIG. 3 is a partial sectional view of the dispensing device of FIG. 1;

FIG. 4a and

FIG. 4b are three-dimensional views of a second embodiment of a device according to the invention for dispensing an eyedrop;

FIG. 5 is a three-dimensional view of an alternative embodiment of the dispensing device of FIG. 1 in the form of a single-dose vial;

FIG. 6 is a three-dimensional view of an alternative embodiment of the dispensing device of FIGS. 4a and 4b;

FIG. 7a and

FIG. 7b and

FIG. 7c are partial sectional views of three alternative embodiments of the device of FIG. 4a, 4b or 6;

FIG. 8a and

FIG. 8b are partial cross-sectional views of a fourth alternative embodiment of the dispensing device of FIG. 4a and 4b or 6;

FIG. 9a is a partial sectional view of a third embodiment of a device according to the invention for dispensing an eyedrop;

FIG. 9b and

FIG. 9c are detailed sectional views of the head of the dispensing device of FIG. 9a;

FIG. 10 is a partial sectional view of an actual implementation of the device of FIG. 7c;

FIG. 11 is a partial sectional view of a practical embodiment of the device of FIG. 7a; and,

FIG. 12 is a partial sectional view of another alternative embodiment of the device according to the invention for dispensing an eyedrop.

For greater clarity, identical or similar elements are identified by identical reference signs in all of the figures.

### DETAILED DESCRIPTION OF AN EMBODIMENT

It will first be noted that, throughout the description, a “device for dispensing an eyedrop” is understood to mean any type of device designed for this purpose: single-dose or multi-dose vials, heads or caps intended to cooperate with independent vials, etc.

Moreover, the following description of the various elements and features of embodiments of a device according to the invention for dispensing an eyedrop is made with said device in a vertical upright position: that is to say, said device in a vertical position, with the dispensing head upward, as illustrated in the figures, thus allowing the patient to place an eyedrop in an eye without tilting the head back.

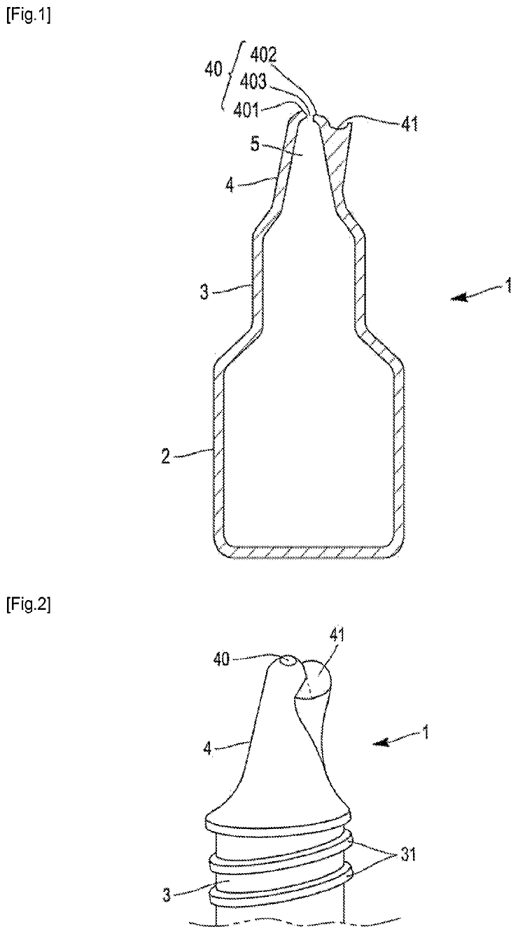

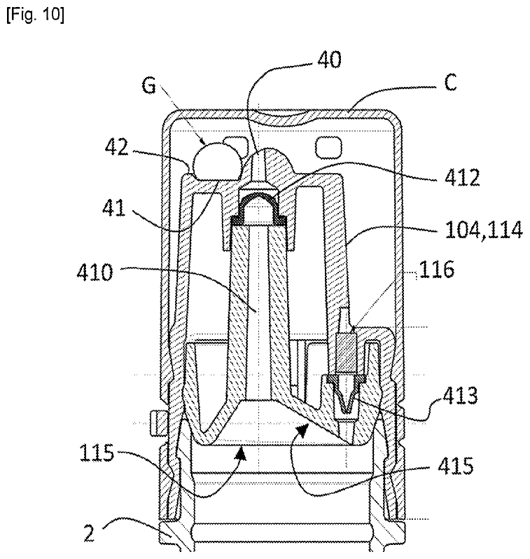

With reference to FIGS. 1 to 3, we will describe a first embodiment of a device 1 according to the invention for dispensing an eyedrop. Here, the device 1 according to the invention for dispensing an eyedrop has, in a vertical position, with a dispensing head being at the top, a flexible vial 2, a head support 3 and a dispensing head 4. The vial 2, the head support 3 and the head 4 are made in one piece as a single component. Here, the head support comprises a thread enabling it to cooperate with a complementary thread of a protective cap for the head 4. The head 4 has at its top, according to the figures, an open delivery end 40 associated with a dispensing surface 41. The dispensing surface 41 is positioned adjacent to the delivery end 40 so that the dispensing surface 41 extends on the side or laterally with respect to the delivery end 40. On the other hand, the dispensing surface 41 extends below the delivery end 40, with respect to the figures (that is to say, the device according to the invention for dispensing an eyedrop being in a vertical position, a dispensing head being at the top). In other words, the delivery end 40 extends above, or projects from, the dispensing surface 41. Preferably, the dispensing surface 41 is substantially concave in shape and has an outer dispensing rim 42 (see FIGS. 10 to 12) opposite the delivery end 40. In an alternative embodiment, the dispensing surface 41 is attached to the dispensing head 4, in particular as a part independent of said dispensing head.

The dispensing head 4 comprises a supply conduit 5, a distal end of which is the delivery end 40. On the opposite side, a proximal end of the supply conduit 5 opens into the vial 2 and has a flared shape. The dispensing end 40 has an opening 403 delimited by a border 401, 402. Here, this border is generally inclined toward the dispensing surface 41, the border having a top edge 401 and a bottom edge 402 diametrically opposite. The bottom edge 402 is adjacent to the dispensing surface 41. Thus, the opening 403 is inclined toward the dispensing surface 41. In addition, this gives an asymmetrical shape to the delivery end 40 with respect to a longitudinal axis of the device 1 according to the invention for dispensing an eyedrop.

With reference to FIG. 5, we will now describe an alternative embodiment of the device 1 according to the invention for dispensing an eyedrop as described above. The device 1 according to the invention for dispensing an eyedrop is a multi-dose version, whereas the alternative embodiment of the device 130 according to the invention for dispensing an eyedrop as shown in FIG. 5 is a single-dose version. Overall, the device 130 according to the invention for dispensing an eyedrop is in one piece and comprises, as previously, in a vertical position, a dispensing head at the top, and a flexible vial 2 surmounted by a dispensing head 134. The dispensing head 134 comprises, at its top, a delivery end 40, which corresponds to the distal end of the supply conduit, and extends above a dispensing surface 141 which surrounds said delivery end 40. The supply conduit of the dispensing head 134, in its proximal part, has a flared shape and forms the junction with the vial 2. The device 130 according to the invention for dispensing an eyedrop further comprises a handling tab 131, known per se, situated under the vial 2.

We will now briefly describe a use of the above-described devices 1 and 130 according to the invention for dispensing an eyedrop. In a first step, the user inverts the device 1, 130 according to the invention for dispensing an eyedrop in order to allow the eyedrop solution to come into contact with the flared shape at the proximal end of the supply conduit 5, this flared shape forming an attachment buffer surface for a predetermined quantity of eyedrop solution. The device 1, 130 according to the invention for dispensing an eyedrop is then once again placed in a vertical position, and a drop of finite and calibrated volume remains attached to the attachment buffer surface of the proximal end of the supply conduit 5. The user then presses on the walls of the flexible vial 2 until the eyedrop appears as a bead at the delivery end 40, which eyedrop then slides over the dispensing surface 41, 141 where it remains. This operation is carried out with the device 1, 131 according to the invention for dispensing an eyedrop in a vertical position, the dispensing head 4, 134 being at the top. While maintaining this vertical position of the device 1, 131 according to the invention for dispensing an eyedrop, the user, keeping their head straight, brings the device to the cheek located under the target eye to be treated, positioning the dispensing rim of the dispensing surface 41, 141 against the edge of the lower eyelid of the target eye. Once the outer rim of the dispensing surface 41, 141 is in contact with the edge of the lower eyelid, the eyedrop present on the dispensing surface 41, 141 “slides” into the conjunctival cul-de-sac of the target eye by capillary action: the eyedrop is as it were aspirated by the fluid present in the conjunctival cul-de-sac.

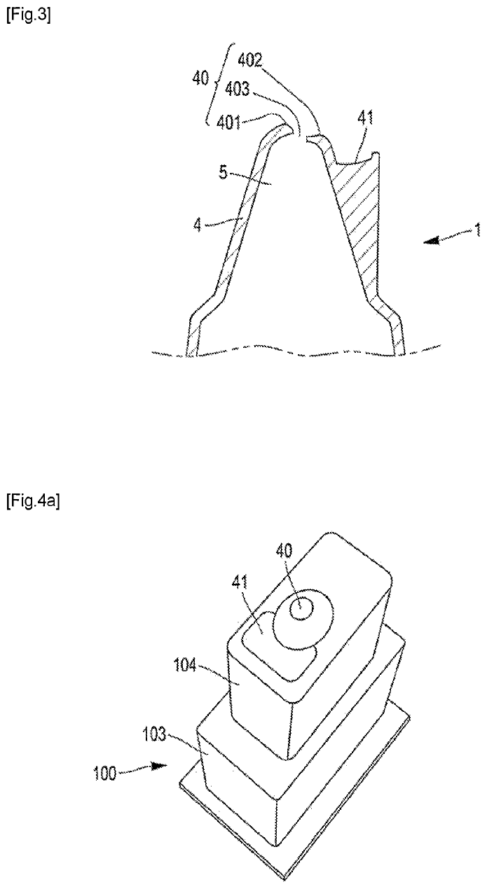

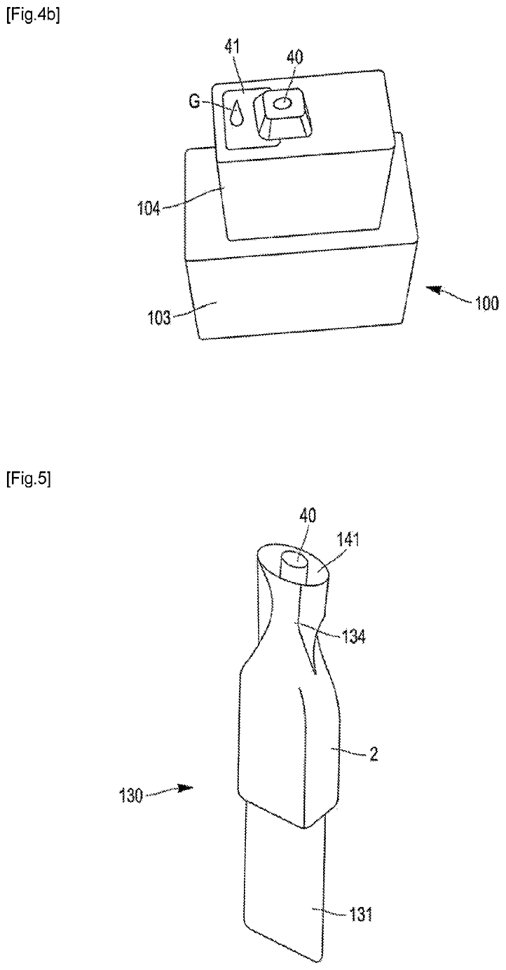

Now, with reference to FIGS. 4a and 4b, we will describe a second embodiment of a device 100 according to the invention for dispensing an eyedrop. It should be noted that FIG. 4b shows the device 100 according to the invention for dispensing an eyedrop from FIG. 4 with an eyedrop present on the dispensing surface 41. The device 100 according to the invention for dispensing an eyedrop is of generally rectangular cross section and is here in the form of a plug intended to be fixed on a vial containing eyedrop solution, which vial may be flexible or rigid. Thus, the device 100 according to the invention for dispensing an eyedrop comprises, in a vertical position, with a dispensing head at the top, a head support 103 of rectangular cross section, surmounted by a dispensing head 104, itself of rectangular cross section, smaller than that of the head support 103. At the top of the dispensing head 104, the device 100 according to the invention for dispensing an eyedrop comprises a dispensing surface 41 adjacent to a delivery end 40 which extends above said dispensing surface 41. As previously, the delivery end 40 has an opening inclined toward the dispensing surface 41. In addition, the inclined opening comprises an inclined surface, of substantially concave shape, at the bottom of which the supply conduit 410 opens out.

In a variant embodiment, the overall cross section of the device 100 according to the invention for dispensing an eyedrop is of circular or oval shape. The oval shape is illustrated in FIG. 6, where the device 110 according to the invention for dispensing an eyedrop comprises a head support 113, and a dispensing head 114 of oval cross section.

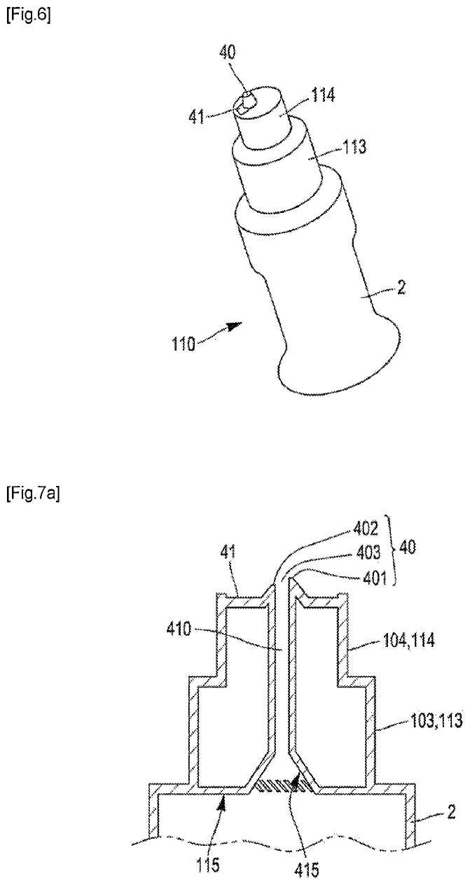

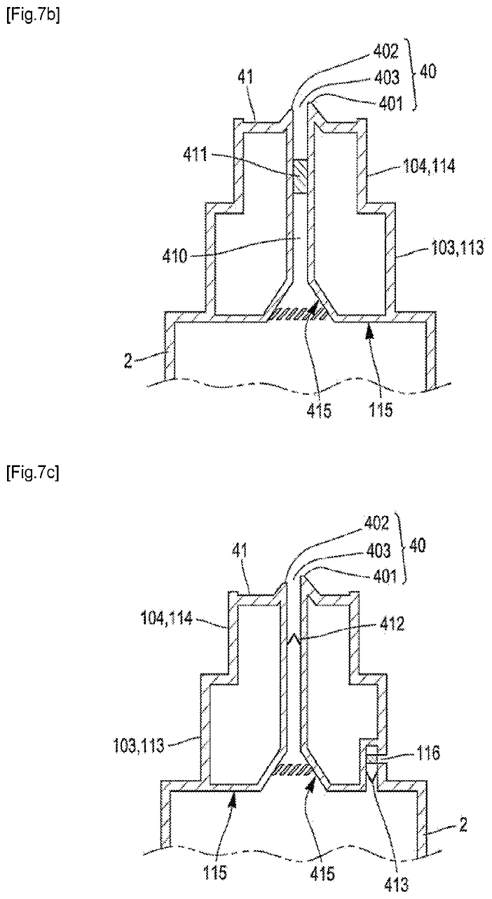

With reference to FIG. 7a, we will describe in more detail the device 100 or 110 according to the invention for dispensing an eyedrop from the preceding FIGS. 4a, 4b and 6. The dispensing head 104, 114 comprises a supply conduit 410 whose distal end has the delivery end 40. On the opposite side, the proximal end of the supply conduit opens into the vial 2 at a surface 115.

The delivery end 40, in this case substantially cone-shaped, comprises an opening 403 delimited by a border 401, 402, as has already been described for the first embodiment of the device 1 according to the invention for dispensing an eyedrop. Here again, this border is generally inclined toward the dispensing surface 41, the border having a top edge 401 and a bottom edge 402 diametrically opposite. The bottom edge 402 is adjacent to the dispensing surface 41. Thus, the opening 403 is inclined toward the dispensing surface 41. In addition, this gives an asymmetrical shape to the delivery end 40 with respect to a longitudinal axis of the device 1 according to the invention for dispensing an eyedrop.

For its part, the proximal end has a concave-shaped attachment buffer surface 415 in the bottom of which the supply conduit 410 opens out. Here, the attachment buffer surface 415 is of frustoconical shape. In general, the attachment buffer surface 415 is of a suitable concave shape: of flared shape, the shape of an ovoid portion, of a prism, etc. This attachment buffer surface 415 is shaped and dimensioned so as to retain, by surface tension, a pre-defined quantity of eyedrop solution that corresponds to the eyedrop that is to be delivered into a patient's eye by the device 100, 110 according to the invention for dispensing an eyedrop. On the other hand, a surface coating of the attachment buffer surface 415 can also be provided for this purpose: the latter may have a surface state having a certain suitable roughness, be covered with a grid, or else with a layer of hydrophilic material compatible with medical use which facilitates the retention of the eyedrop in a volume delimited by the attachment buffer surface 415.

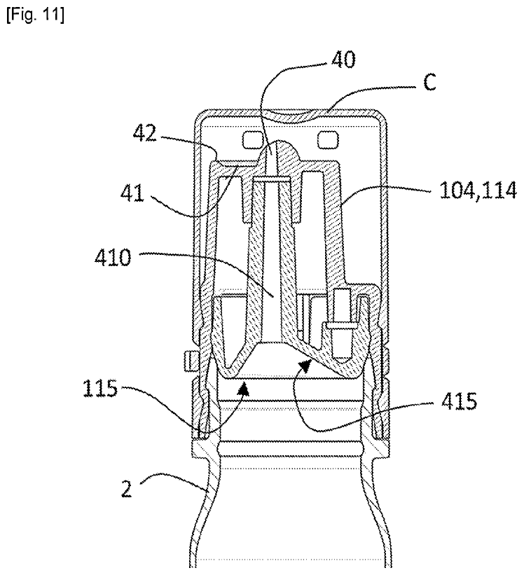

FIG. 11 illustrates an actual implementation of the device according to the invention for dispensing an eyedrop which has just been described with reference to FIG. 7a. A protective cap C is shown in FIG. 11.

As has been indicated above, the dispensing surface 41 is of substantially concave shape and has an outer dispensing rim 42 opposite the delivery end 40. The dispensing surface 41 can have a flat bottom as illustrated, or else is rounded in shape in the form of a portion of an ovoid or a sphere.

During use, the patient or person using the device 100, 110 according to the invention for dispensing an eyedrop puts the device 100, 110 according to the invention for dispensing an eyedrop in a head-to-tail position: with the dispensing head 104, 114 then at the bottom, the eyedrop solution contained in the vial 2 covers the surface 115, and the attachment buffer surface 415 is covered by said eyedrop solution. The user or the patient then returns the device 110, 110 according to the invention for dispensing an eyedrop to the vertical position, the dispensing head 104, 114 being positioned at the top. In this position, the eyedrop solution falls back to the bottom of the vial, and a predetermined quantity of eyedrop solution is retained by the attachment buffer surface 415. Consequently, a volume of air is positioned in the vial between the remaining eyedrop solution and said predetermined quantity of eyedrop solution retained by the attachment buffer surface 415. The user or patient then squeezes the vial 2. The pressure established in the vial 2 then pushes the predetermined quantity of eyedrop solution from the attachment buffer surface 415 into the supply conduit 410 toward the delivery end 40, where the eyedrop G will then appear as a bead and slide over the dispensing surface 41. While maintaining the vertical position of the device 100, 110 according to the invention for dispensing an eyedrop, with the dispensing head at the top, the user, while keeping their head straight, brings the device to the cheek located under the target eye to be treated, positioning the dispensing rim 42 of the dispensing surface 41 against the edge of the lower eyelid of the target eye. Once the outer rim of the dispensing surface 41 is in contact with the edge of the lower eyelid, the eyedrop G present on the dispensing surface 41 “slides” into the conjunctival cul-de-sac of the target eye by capillary action: the eyedrop G is as it were aspirated by the fluid present in the conjunctival cul-de-sac.

In an alternative embodiment, provision may be made for the vial 2 to be rigid with a delimited flexible surface, so as to easily control an ejection of the eyedrop from the attachment buffer surface 415 via the supply conduit 410 until it slides from the delivery end 40.

In a first alternative embodiment illustrated in FIG. 7b, the supply conduit 410 comprises a bactericidal or antibacterial filter 411.

In a second alternative embodiment illustrated in FIG. 7c, the supply conduit comprises a non-return valve 412. In addition, the head support 103, 113 comprises a conduit 116 for air intake into the vial 2. This air intake conduit 116 is equipped with a non-return valve 413 upstream of which is situated a bactericidal or antibacterial filter.

FIG. 10 illustrates an actual implementation of this second alternative embodiment of the device according to the invention for dispensing an eyedrop.

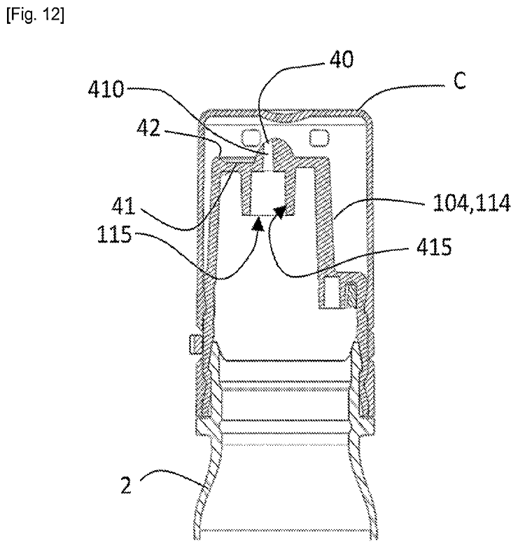

With reference to FIG. 12, we will describe another alternative embodiment of a device according to the invention for dispensing an eyedrop. This alternative differs from the embodiments described above by the fact that the supply tube 410 is very short. Indeed, a length of this supply tube 410 is of the order of one thickness of the dispensing head at the delivery end 40, the surface 115 and the attachment buffer surface 415 being positioned as close as possible to said delivery end 40. This makes it possible to obtain a dispensing head that is optimized in terms of the volume of material and the number of components used for its manufacture.

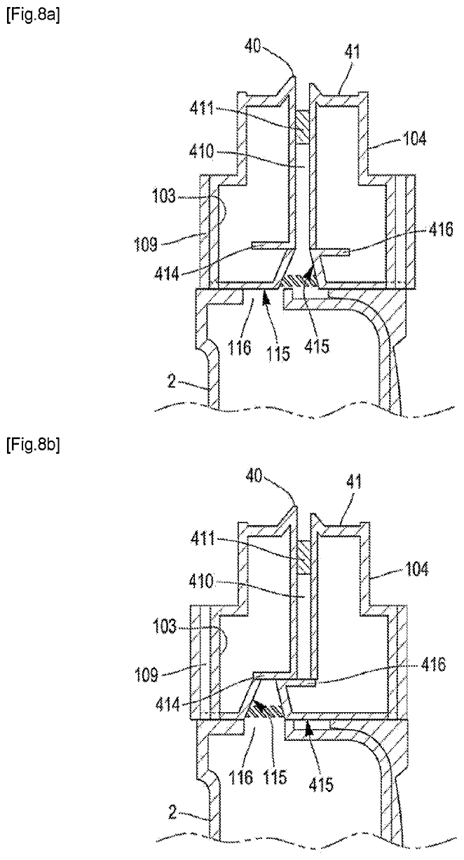

In a third variant illustrated in FIGS. 8a and 8b, the device 100, 110 according to the invention for dispensing an eyedrop comprises, at the level of the head support 103, a drawer 109 sliding, perpendicularly to the longitudinal axis of the device 100, 110 according to the invention for dispensing an eyedrop, between an open dispensing position illustrated in FIG. 8a and a closed position illustrated in FIG. 8b. In an alternative embodiment, the drawer 109 pivots about an axis of rotation parallel to a longitudinal axis of the device 100, 110 according to the invention for dispensing an eyedrop. The drawer 109 comprises the surface 115 and also the attachment buffer surface 415 provided with a flange 416. The supply conduit comprises a symmetrical flange 414. In the open dispensing position, the volume delimited by the attachment buffer surface 415 is fluidically connected to the supply conduit 410 for ejection of an eyedrop toward the dispensing surface 41. The attachment buffer surface 415 is cut from the vial 2. In the closed position of the drawer 109, the attachment buffer surface 415 is displaced so as to be isolated from the supply conduit 410 and to be fluidically connected to the vial 2. In this position, the flange 414 of the supply conduit closes the top of the attachment buffer surface 415, and the flange 416 closes off the supply conduit 410. In this closed position, by turning upside down the device 100, 110 according to the invention for dispensing an eyedrop, the volume delimited by the attachment buffer surface 415 is filled with a predetermined quantity of eyedrop solution. The use is also the same, mutatis mutandis.

In an alternative embodiment (not illustrated) to all of the previously described embodiments, the supply conduit comprises a distal portion inclined with respect to a longitudinal axis of the device, the dispensing end being positioned on the inclined distal portion.

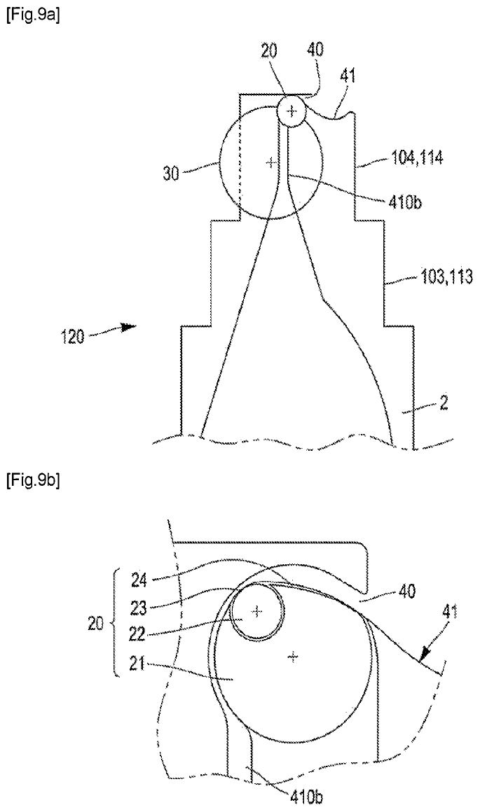

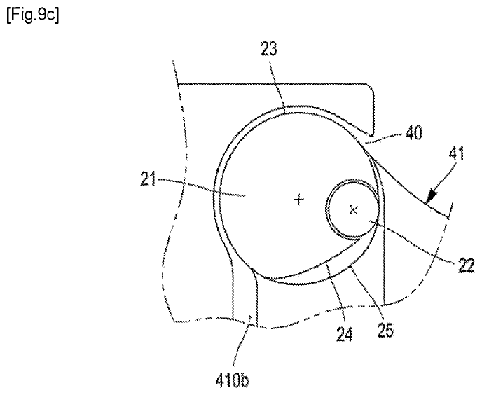

With reference now to FIGS. 9a to 9c, we will describe a third embodiment of a device 120 according to the invention for dispensing an eyedrop. This embodiment differs from the above-described second embodiment by the presence of means forming a delivery pump 20, 30. This device 120 according to the invention for dispensing an eyedrop comprises a vial 2 having a reservoir bag of eyedrop solution directly connected fluidically to the pump-forming means 20, 30 via a flexible supply conduit 410b.

At the level of the delivery head 104, 114, the device 120 according to the invention for dispensing an eyedrop comprises a delivery wheel 30 making it possible to use a roller 21. In a variant embodiment, the wheel 30 can be positioned at the head support 103, 113 of the device 120 according to the invention for dispensing an eyedrop. The roller 21 comprises an eccentric 22 which crushes a flexible tube 23 extending the supply conduit toward the delivery end 40 overhanging the dispensing surface 41. The roller 21, driven in rotation by the wheel 30, is mounted so as to be able to rotate about its axis in a circularly cylindrical housing 25. In addition, the roller 21 comprises a kind of flat edge 24 upstream of the eccentric 22, the roller 21 turning clockwise in FIGS. 9a to 9c. The assembly works like a peristaltic pump.

The presence of the flat edge 24 makes it possible to dose the quantity of eyedrop solution that the pump will eject through the delivery end 40 toward the dispensing surface 41. In fact, in the closed position of non-use illustrated in FIG. 9c, the roller 21 completely crushes the flexible tube 23, isolating the supply conduit 410b from the opening of the delivery end 40. In the operating position, as partly illustrated in FIG. 9b, the eccentric 22 pushes the quantity of eyedrop solution defined by the flat edge 24 along the flexible conduit 23 toward the delivery end 40.

In use, once the user has generated the eyedrop to be administered on the dispensing surface 41 via the wheel 30, the procedure is similar to the one already described above for the embodiments of the device according to the invention for dispensing an eyedrop. Such a structure of an eyedrop dispenser as described above, and illustrated by FIGS. 1 to 12, makes it possible to dose and dispense a single eyedrop. Existing devices of this type do not provide such safety in the dispensing of a single dosed drop.

In all of the embodiments, the dispensing surface 41, 141 is concave in shape, like a spoon, or flat and is delimited, opposite the delivery end 40, by a dispensing rim 42. In general, the dispensing surface 41, 141 is shaped so as to facilitate capillary transfer of the eyedrop, which is present on said dispensing surface 41, 141, into the conjunctival cul-de-sac of the target eye, while leaving no aqueous residues on the dispensing surface 41, 141. To this end, the dispensing surface 41, 141 can be covered by or made of a hydrophobic material.

Of course, it is possible to make numerous modifications to the invention without thereby departing from the scope of the invention.

1. A device for dispensing an eyedrop, comprising a dispensing head having a supply conduit for eyedrop solution, the supply conduit having an open delivery end and a dispensing surface in proximity to the delivery end, wherein, with the device in a vertical position with the dispensing head upward, the dispensing surface extends on at least one side of and below the delivery end, the delivery end being arranged to generate the eyedrop toward the dispensing surface.

2. The device as claimed in claim 1, wherein the delivery end is asymmetrical with respect to a longitudinal axis of the device.

3. The device as claimed in claim 2, wherein the delivery end has an opening inclined toward the dispensing surface.

4. The device as claimed in claim 3, wherein the inclined opening has an inclined surface.

5. The device as claimed in claim 2, wherein the supply conduit has a distal portion inclined with respect to a longitudinal axis of the device, the delivery end being positioned on the inclined distal portion.

6. The device as claimed in claim 1, wherein the supply conduit comprises an attachment buffer surface situated at the proximal end and arranged so as to permit retention of a drop and to calibrate the drop.

7. The device as claimed in claim 1, wherein the supply conduit has means forming a delivery pump.

8. The device as claimed in claim 7, wherein the means forming a delivery pump comprises a peristaltic pump.

9. The device as claimed in claim 1, wherein the supply conduit comprises a non-return valve.

10. The device as claimed in any one of claims 1 to 9, characterized in that the supply conduit comprises an antibacterial and/or bactericidal filter (411).

11. The device as claimed in claim 1, wherein the dispensing surface comprises a dispensing rim opposite the delivery end.

12. The device as claimed in claim 11, wherein the dispensing surface has a flat bottom, in the form of an ovoid portion or a portion of a sphere.

13. The device as claimed in claim 1, wherein the dispensing surface is hydrophobic.
